(19) United States
(12) Patent Application Publication
(10) Pub. No.: US 2026/0041811 A1
(43) Pub. Date: Feb. 12, 2026

(54) HYDROGEL FOR THE REDUCTION OF BIOFILMS

(71) Applicant: PAUL HARTMANN AG, Heidenheim (DE)

(72) Inventors: Markus Johannes KETTEL, Heidenheim (DE); Alena JANSSEN, Veitshöchheim (DE); Jonathan NOSWORTHY, Neu-Ulm (DE)

(73) Assignee: PAUL HARTMANN AG, Heidenheim (DE)

(21) Appl. No.: 18/995,725

(22) PCT Filed: Jul. 14, 2023
(86) PCT No.: PCT/EP2023/069726

§ 371 Date: Jan. 16, 2025

(30) Foreign Application Priority Data
Jul. 18, 2022 (DE) 10 2022 117 861.7

(51) Int. Cl. A61L 15/60; A61L 15/26; A61L 15/42; A61L 15/44

(52) U.S. Cl. CPC A61L 15/60; A61L 15/26; A61L 15/425; A61L 15/44; A61L 2300/102; A61L 2300/406

(57) ABSTRACT

The present invention relates to hydrogels suitable for medical use—especially in wound treatment. The hydrogels contain a metal complex as active ingredient and display excellent antimicrobial action against biofilms. Furthermore, the hydrogels assure an excellent release of moisture and create optimal prerequisites for a rapid healing process even in the case of infected wounds. Further aspects of the invention relate to a process for producing the hydrogels and to wound dressings that contain the hydrogel as a constituent.

### FIELD OF THE INVENTION

The present invention relates to a hydrogel for reduction of biofilms, comprising an active ingredient embedded in the hydrogel, where the hydrogel is notable for high release of moisture and active ingredient. The hydrogel is suitable for medical use. The invention further relates to a process for producing the hydrogel, to the use of the hydrogel for medical purposes and to a wound dressing containing the hydrogel as wound contact layer.

### BACKGROUND AND OBJECT OF THE INVENTION

It is common knowledge in the field of wound treatment that a moist wound environment promotes healing. The cause of this is accelerated epithelialization, reduced scarring and lowered inflammation reaction by virtue of the moist wound environment. Moist wound treatment is suitable in particular for poorly healing, chronic and infected wounds. Specifically in the latter case, colonization of the wound by microorganisms can result in complications. In general, an infection of the wound is attributable to bacteria. Measures for removal or killing of the bacteria are not always successful since the bacteria frequently adapt to the altered conditions. This especially comprises the development of resistances and the consolidation to form biofilms. While modern wound treatment has already reacted to the development of resistances and topical application of conventional antibiotics has declined, biofilms in wounds are still a serious problem and are a subject of current research. WO2017191453 describes the use of metal complexes for controlling biofilms in wounds. Additionally disclosed are hydrogels containing such metal complexes. Said hydrogels have been produced from a particular polyacrylic acid—of the Carbopol® brand name. Even though Carbopol® is a popular raw material in polymer chemistry, it is associated with drawbacks in practical use. For instance, the use of Carbopol® leads to dust formation. It is also difficult to bring the powder into solution by stirring because of its low density. This leads to long mixing times, in which there can be unwanted foaming. Moreover, in the case of (intentional or unintentional) contact with moisture, there can be formation of lumps.

In an animal model, Carbopol® showed inhibiting effects on wound healing (Grip, Jostein, et al. “Sprayable Carbopol hydrogel with soluble beta-1, 3/1, 6-glucan as an active ingredient for wound healing-development and in-vivo evaluation.” European Journal of Pharmaceutical Sciences 107 (2017): 24-31). There is thus a need for hydrogels that contain active ingredients against biofilms, release these into the wound to a high degree together with moisture, and have a substrate that does not hinder wound healing. It is an object of the present invention to cover this need. The object is achieved by a process as claimed in claim 1, a hydrogel as claimed in claim 9, and a wound dressing as claimed in claim 15.

### Definitions

The term “hydrogel” in the context of the present invention refers to a finely dispersed system composed of at least one solid phase and one liquid phase. This solid phase forms a spongelike three-dimensional network, the pores of which are filled by a liquid (lyogel). The two phases penetrate one another. The two phases preferably fully penetrate one another. As a result of water absorption, the three-dimensional network can increase its volume through swelling without losing structural cohesion. The term “hydrogel” is also referred to synonymously hereinafter as hydrogel composition or hydrogel matrix. By varying the reaction parameters of the production process of the invention, the resulting hydrogel may sometimes have a foamlike character. Such products are also referred to hereinafter as hydrogels of the invention. What is crucial is that they can be obtained by the production process of the invention.

The term “active ingredient” in the context of the present invention may have the following meanings:
a) the Ag₂Zn(EDTA) complex compound,
b) an aqueous solution of said complex compound or
c) an aqueous solution of the starting substances silver nitrate, zinc sulfate or zinc sulfate monohydrate and tetrasodium EDTA, which combine to form said complex. Unless stated otherwise, the term “active ingredient” may be understood to mean primarily the complex compound mentioned in a). The active ingredient has both biofilm-reducing and antibacterial properties, and additionally promotes the release of moisture from the hydrogels of the invention.

The term “reaction mixture” means a mixture of all constituents for the running of the process of the invention for production of a biofilm-reducing hydrogel. This includes all reactants, and may also optionally include optional auxiliaries such as consistency modifiers, stabilizers, acids, alkalis etc. Various embodiments of the process of the invention are elucidated hereinafter. The composition of the reaction mixture may depend on the particular embodiment.

The term “biofilm” means a thin, two-dimensionally spread mucosal film in which there are populations of microorganisms. The mucosal film is formed by the microorganisms and is a matrix of extracellular polymeric material that includes the microorganisms. The populations of microorganisms are typically mixed populations. Biofilms form on surfaces, which can also include wound tissue. The microorganisms organized in the biofilm show elevated resistance to conventional antibiotics, to disinfectants and to the immune system of advanced forms of life.

The expression “biofilm-reducing” encompasses both the killing of bacteria (disinfecting properties) and the breaking of chemical bonds, and the removal of chemical binding partners from the biofilm-forming extracellular matrix. The latter generally lowers the defenses of the microorganisms previously organized in the biofilm against effects such as disinfectants, antibiotics, the immune system or environmental influences. “Biofilm-reducing” in the context of the present invention means only those substances that are suitable for application to open wounds without displaying any noticeable harmful effect on the individual to be treated.

The term “starting solution” means a solution containing at least water, an amine-terminated prepolymer and the active ingredient. Further constituents, for example a polyhydric alcohol, preferably glycerol, or a base, preferably ammonia, may optionally be present.

The terms “moisture delivery” and “moisture release” mean the same thing.

The expression “colony-forming unit” (CFU) means a single division-capable cell of a unicellular organism, especially a unicellular organism or bacterium that is pathogenic to humans.

### DESCRIPTION OF THE INVENTION

The hydrogels of the invention have excellent properties with regard to their ability to store water and release it to a wound. It has been found that the active ingredient content in the hydrogel, i.e. the concentration of the complex in the hydrogel, has an influence on the extent to which moisture is released to the wound by the hydrogel. Moisture release increases with increasing concentration of active ingredient in the hydrogel.

The moisture released to an increased extent from the hydrogel of the invention by the active ingredient has the advantage that active ingredient is released into the wound together with the moisture.

Measurements have shown that, surprisingly, the bond strength of the hydrogel of the invention can be enhanced by the embedding of the active ingredient. At the same time, the atraumatic character of the hydrogel as wound contact layer is maintained, since the hydrogel does not stick to the wound. The embedding of the active ingredient into the hydrogel can thus under some circumstances permit further adhesive layers or a circumferential adhesive edge to be made smaller or—depending on the end use of the wound dressing—to be dispensed with entirely. This facilitates the production of such a wound dressing of the invention and saves costs. During the development of the present invention, it has been found that the bond strength of the hydrogel increases with increasing proportion of the active ingredient in the hydrogel, meaning that the more Ag₂Zn(EDTA) is present in the hydrogel, the stronger its adhesion to the skin or wound. This is attributed at least partly to the fact that the degree of crosslinking within the hydrogel matrix declines with increasing active ingredient content. In this context, the invention also encompasses the use of an Ag₂Zn(EDTA) complex in a hydrogel in order to adjust or to enhance the bond strength of the hydrogel. In particular, it is possible to utilize the use in order to adjust the bond strength of the hydrogel on tissue such as skin or wound tissue.

The process of the invention for production of a hydrogel comprises the following steps:
i. providing an isocyanate-terminated prepolymer containing polyalkylene oxide units,
ii. providing an amine-terminated prepolymer containing polyalkylene oxide units,
iii. dissolving the substance provided in ii in a water-containing liquid in order to obtain an aqueous formulation,
iv. mixing the aqueous formulation and a solution, especially an aqueous solution, containing an Ag₂Zn(EDTA) complex, in order to obtain a starting solution,
v. combining the starting solution and the isocyanate-terminated prepolymer to give a reaction mixture, whereby these are converted by polymerization to the hydrogel,
   wherein the pH of the solution from step iv is at least 9, preferably 10 to 12, and wherein the reaction mixture from step v does not contain any acrylic acid or polyacrylic acid.

In the last step v, the isocyanate-terminated prepolymer from step i and the amine-terminated prepolymer from step ii that was present in the starting solution are reacted with one another. Further reactants such as a polyhydric alcohol may likewise be present. Polymerization takes place during the reaction, meaning that the two prepolymers form a crosslinked gel matrix in the course of a polymerization. Water and the Ag₂Zn(EDTA) complex are taken up here into the gel matrix, but can be released again therefrom. As a result of the reaction, the active ingredient-containing hydrogels of the invention are obtained.

The starting solution and the isocyanate-terminated prepolymer may be combined gradually—i.e. a little at a time. It is possible to combine the starting solution and the isocyanate-terminated prepolymer in a casting mold. On conclusion of the reaction, the finished hydrogel can be removed from the casting mold and can retain the shape defined by the casting mold. The Ag₂Zn(EDTA) complex compound is the biofilm-reducing active ingredient within the hydrogel and is obtainable by dissolving the following compounds in a polar, preferably aqueous liquid, especially water: tetrasodium EDTA, silver nitrate and zinc sulfate/zinc sulfate monohydrate. For the preparation of Ag₂Zn(EDTA), it is possible first to allow polar, preferably aqueous solutions of tetrasodium EDTA (for example a 3.8% solution) and silver nitrate (for example a 6.8% solution) to react with one another, and to filter out the precipitated solid. The filtrate may be added to a polar, preferably aqueous solution, of zinc sulfate monohydrate (for example a 1.9% solution). After further filtration, the Ag₂Zn(EDTA) complex is obtained.

The process of the invention and the associated chemical reactions can proceed at room temperature. In the case of addition of alcohols to the reaction mixture, a lower temperature can sometimes lead to better results, which is elucidated in detail elsewhere.

The reaction mixture from the process of the invention and the hydrogel of the invention do not contain any acrylic acid or polyacrylic acid. It is inadvisable to add acrylic acid since this could exert an adverse effect on wound healing.

In the production process of the invention, the mass ratio of the isocyanate-terminated prepolymer to the amine-terminated prepolymer may be between 1.3 and 3.2, preferably between 1.4 and 1.8, more preferably between 1.5 and 1.7 and most preferably between 1.55 and 1.65.

In the hydrogels mentioned, the solid phase need not necessarily be formed solely by a polymer that forms from the reaction between an amine-terminated prepolymer and an isocyanate-terminated prepolymer. The reaction may likewise involve a polyhydric alcohol, the free hydroxyl groups of which can react with isocyanate groups. The polyhydric alcohol component contributes to additional crosslinking, which, especially in the case of polyhydric alcohols having more than two hydroxyl groups, leads to three-dimensional crosslinking of the prepolymers. The reaction between a polyhydric alcohol and an isocyanate group gives rise to a carbamic ester, also referred to as a urethane. This reaction can be accelerated by acids or bases as catalyst, and reversed by supply of thermal energy. It is possible to perform the process of the invention at room temperature. If the reaction temperature is kept constant between 5° C. and 30° C., preferably between 5° C. and 20° C., this reaction can be effected in a sufficient ratio to obtain hydrogels with covalently incorporated polyhydric alcohols that have advantageous properties. In this context, the reaction mixture may contain at least one polyhydric alcohol. The latter may be selected from the group of the dihydric, trihydric, tetrahydric, pentahydric or hexahydric alcohols. In particular, the alcohol may be selected from the group of the glycols, especially ethylene glycol, polyethylene glycols having a mass of 200 g/mol to 6000 g/mol, preferably polyethylene glycols having a mass of 300 g/mol to 2000 g/mol, and sorbitol or glycerol or mixtures thereof. These alcohols are additionally of excellent suitability as moisture donors and thus constitute a care component for the skin surrounding the wound. Hydrogels containing one or more of these alcohols as partners in the above-described reaction have a high absorption capacity for wound exudate and a reduced moisture loss. They have a bond strength that allows atraumatic changing of dressings. Because of their low cytotoxicity, they are of very good compatibility for wound tissue. Moreover, such gels are capable of concentrating growth factors in the wound exudate that are necessary for wound healing and hence of accelerating wound healing. The presence of polyalcohol in the wound leads to slowed evaporation of moisture, which keeps the wound moist for longer.

Therefore, in a preferred embodiment of the process of the invention, glycerol is added to the reaction mixture. The glycerol may be added to the water-containing liquid from step iii of the process. Accordingly, it is preferably a feature of the process that the water-containing liquid in step iii contains glycerol.

The amount of glycerol added is preferably chosen such that the reaction mixture in step v of the process contains 10% to 30% by weight of glycerol.

The hydrogels thus produced have particularly advantageous properties with regard to cell compatibility, liquid loss and adhesion capacity.

In a particularly preferred embodiment, the polyhydric alcohol used is glycerol in a concentration of 15-25% by weight. The hydrogels thus produced also have a particularly high absorption capacity.

In another embodiment, the polyhydric alcohol used is ethylene glycol in a concentration of 5-30% by weight, preferably 10-25% by weight, more preferably 15-20% by weight. Such hydrogels have advantageous properties with regard to moisture loss, absorption capacity and cell compatibility.

In a further embodiment, the polyhydric alcohol used is sorbitol in a concentration of 5-30% by weight, preferably 10-25% by weight, more preferably 15-20% by weight. Such hydrogels have advantageous properties with regard to absorption capacity and cell compatibility.

In a further embodiment, the polyhydric alcohol used is PEG300 (polyethylene glycol having an average relative molecular mass of 300 g/mol) in a concentration of 5-30% by weight, preferably 10-25% by weight, more preferably 15-20% by weight. Such hydrogels have advantageous properties with regard to moisture loss and absorption capacity.

In a further embodiment, the polyhydric alcohol used is PEG2000 (polyethylene glycol having an average relative molecular mass of 2000 g/mol) in a concentration of 5-30% by weight, preferably 10-25% by weight, more preferably 15-20% by weight. Such hydrogels of the invention have advantageous properties with regard to cell compatibility.

In the process of the invention for production of a biofilm-reducing hydrogel, the reaction mixture may thus comprise a polyalcohol, especially glycerol. The reaction mixture may preferably contain 10% to 30% by weight of polyalcohol such as glycerol. The reaction mixture more preferably contains 15% to 25% by weight of polyalcohol such as glycerol. The reaction mixture most preferably contains 18% to 23% by weight of polyalcohol such as glycerol. The term “polyalcohol” also includes a mixture of different polyalcohols. These include, as well as glycerol, the already aforementioned alcohols ethylene glycol, sorbitol, PEG300, PEG2000. Preference is given here to combining one of the above alcohols with glycerols such that the two alcohols make up 10% to 30% by weight of the reaction mixture in the process of the invention.

During the development of the present invention, it has been found that the active ingredient-containing solution which is used in the process of the invention for production of the hydrogel can be stabilized by an alkaline pH. At neutral pH and acidic pH values, by contrast, the chemical components can precipitate out of the solution. The probability of precipitation can be reduced when the pH of the active ingredient-containing solution is adjusted to a pH of at least 9. Consequently, the pH of the active ingredient-containing solution, according to the invention, is at least 9, preferably 10 to 12. The probability of precipitation is additionally also dependent on the concentration of the active ingredient in the solution. A higher concentration makes precipitation more probable. It is preferable to establish a pH of the active ingredient-containing solution of at least 9.5 over and above an active ingredient concentration of 1% Ag₂Zn(EDTA), a pH of at least 10 over and above an active ingredient concentration of 1.5% Ag₂Zn(EDTA), a pH of at least 10.5 over and above an active ingredient concentration of 2% Ag₂Zn(EDTA), and a pH of at least 11 over and above an active ingredient concentration of 2.5% Ag₂Zn(EDTA).

The pH is preferably adjusted using ammonia and/or sodium hydroxide. The use of ammonia is preferred since the best and most stable hydrogels were obtained here. It has been found that, when NaOH is used, the sodium ions compete with the silver present in the Ag₂Zn(EDTA). If required, acetic acid can be added to lower a pH that is too high.

The active ingredient-containing solution is preferably an aqueous solution. The water here has the advantage that it is intercalated into the hydrogel matrix in the course of the reaction and can then be released to the wound.

It is emphasized at this point that the pH of the active ingredient-containing solution need not correspond to the pH of the reaction mixture or of the hydrogel of the invention obtained at a later stage. The hydrogel may also have a neutral or slightly acidic pH without occurrence of precipitation of the active ingredient present.

It is generally known from the prior art to provide wound dressings for moist wound treatment that release an isotonic solution to the wound. In the context of this present invention, it has been found that most of these solutions are disadvantageous in connection with the hydrogel of the invention. The reason is that these solutions generally contain chloride ions. As has been found, the chloride ions enter into a reaction with the silver present in the Ag₂Zn(EDTA) and form sparingly soluble silver chloride. In a preferred embodiment of the process of the invention, the reaction mixture does not contain any chloride ions or chlorine salts—in particular any NaCl. Accordingly, it is then also the case that the hydrogel of the invention, or the hydrogel obtainable by the process of the invention, preferably does not contain any chloride ions or chlorine salts.

The reaction mixture and/or the hydrogel of the invention preferably do not contain any triethanolamine. Addition of triethanolamine is inadvisable since it could have a disruptive effect on the reaction in the process of the invention. The triethanolamine would be able to compete with the amine-terminated prepolymer present in the reaction mixture. Consequently, it is also inadvisable to use triethanolamine to adjust the pH of the hydrogel or the active ingredient-containing solution.

The process of the invention is typically characterized in that the amine-terminated prepolymer is at least partly covalently bound in the reaction in process step v. In one embodiment of the process of the invention, at least 70% by weight of the amine-terminated prepolymer used in the reaction mixture is covalently bound in the production process of the invention, preferably at least 80% by weight of the amine-terminated prepolymer used is covalently bound, more preferably at least 90% by weight of the amine-terminated prepolymer used is covalently bound, and most preferably at least 95% by weight of the amine-terminated prepolymer used in the reaction mixture is covalently bound.

The reaction mixture may contain 0.5% to 4% by weight of the Ag₂Zn(EDTA) complex. The proportion of the complex in the reaction mixture affects the amount of the active ingredient in the resulting hydrogel. In addition, different amounts of active ingredient in the hydrogel affect the efficacy toward microorganisms, biofilms and the release of moisture from the hydrogel. The reaction mixture preferably contains 0.5% to 3.5% by weight of the Ag₂Zn(EDTA) complex. The reaction mixture more preferably contains 1% to 3% by weight of the Ag₂Zn(EDTA) complex. The reaction mixture most preferably contains 1% to 2% by weight of the Ag₂Zn(EDTA) complex.

In a preferred embodiment, the sum total of the masses of amine-terminated prepolymer and isocyanate-terminated prepolymer is 10% to 30% by weight of the reaction mixture.

The reaction mixture may contain the isocyanate-terminated prepolymer in an amount of 10-25% by weight. The reaction mixture preferably contains 11% to 23% by weight, more preferably 12% to 20% by weight, most preferably 13% to 18% by weight, of the isocyanate-terminated prepolymer.

In addition, the reaction mixture may contain the amine-terminated prepolymer in an amount of 3% to 15% by weight, preferably 5% to 15% by weight, more preferably 6% to 12% by weight, most preferably 7% to 10% by weight.

The polyalkylene oxide units of the two aforementioned prepolymers may be formed by polyethylene oxide and/or polypropylene oxide units, where the weight ratio of ethylene oxide to propylene oxide units is preferably 3:1 to 7:1. In addition, the isocyanate-terminated prepolymer advantageously has at least three-arm branching. In particular, the isocyanate-terminated prepolymer has exactly three-arm branching.

A possible isocyanate-terminated prepolymer having aliphatic isocyanate groups is, for example, a three-arm copolymer of propylene glycol and ethylene glycol units that has been reacted terminally in each case with one molecule of isophorone diisocyanate. It typically has a content of reactive isocyanate end groups (NCO groups) of 3.0% to 3.4%, preferably 3.2%, and a molar ratio of ethylene oxide units to propylene oxide units of 3:1 to 4:1. The isocyanate-terminated prepolymer may be Aquapol®. This chemical may be sourced under the Aquapol PI-13000-31 trade name from the manufacturer Carpenter (Richmond, USA).

A possible amine-terminated prepolymer is, for example, a triblock polymer composed of two propylene glycol units, ethylene glycol units and propylene glycol units again, where the polymer has been terminally amine-functionalized in each case with 2-aminopropyl groups. It typically has a content of reactive amine end groups of 0.9554 mmol/g with a molecular mass of on average about 2000 g/mol and a dispersity of 1.08, measured by gel permeation chromatography, and a molar ratio of ethylene units to propylene units of 3:1 to 7:1, preferably 39:6. Such an amine-terminated prepolymer may be sourced under the Jeffamine® ED-2003 name from the manufacturer Huntsman (Everberg, Belgium).

In addition, the invention encompasses a hydrogel having biofilm-reducing properties, obtainable or obtained by the process of the invention.

The hydrogels of the present invention may contain, as a solid phase, a polymer having polyurethane and polyurea groups. As a liquid phase, they may especially contain water and optionally a polyhydric alcohol. Propylene glycol may be excluded therefrom, since this substance is less cell-compatible than glycerol for example.

The hydrogels are suitable for treatment of wounds. The hydrogel may be used for wound treatment in different ways. The gel may first be applied to the wound and then optionally covered with a wound dressing. The wound dressing here may fix the hydrogel and shield it from outside influences. The hydrogel may additionally also be left uncovered on the wound. This is possible particularly in the case of relatively short application periods and in areas of the skin where slippage of the hydrogel is unlikely. Uncovered application enables the particularly rapid inspection of the wound and of the progression of healing.

Water-containing hydrogels used in the context of the present application may especially be hydrogels that form a coherent, discrete layer and do not release any water under a pressure that occurs when the hydrogel is used as intended. For this purpose, the water content in the reaction mixture of the production process should be adjusted correspondingly. Depending on the proportions of the other reactants, the reaction mixture may contain a water content of 40% by weight or more. The reaction mixture preferably contains a water content of 40% to 65% by weight, more preferably 50% to 65% by weight, most preferably 50% to 60% by weight. Accordingly, hydrogels of the invention may have a water content of at least 40% by weight, preferably 40% to 65% by weight, more preferably 50% to 65% by weight and most preferably 50% to 60% by weight.

Hydrogels of the invention generally have a pH that is between 6.5 and 9.5, preferably between 6.5 and 8, more preferably between 7 and 8, and hence covers an optimal spectrum for treatment of infected wounds. It is a particular advantage of the hydrogels of the invention that the hydrogels obtainable by the production process of the invention generally have an optimal pH immediately.

Even though an acidic pH is frequently regarded as optimal for the promotion of wound healing, it is now known that this is not always the case for infected wounds. Depending on the pathogens present in the wound, a basic pH of the hydrogel to be used or of the wound dressing may be advantageous. If an exact adjustment of the pH of the hydrogel of the invention should be desired, this can be effected by addition of suitable substances such as ammonia or acetic acid.

A neutral or slightly acidic pH is recommended for prevention of infections or in the case of only light colonization of the wound. An alkaline pH (pH>7) is recommended when there is already significant colonization of the wound with pathogens. There are pointers that numerous bacterial enzymes and toxins are inhibited in an alkaline medium. If a combination of the wound dressing of the invention or of the hydrogel with antibiotics should be the aim, an alkaline pH is likewise recommended, since most antibiotics are inhibited by an acidic medium.

The hydrogel of the invention can reduce biofilms containing Gram-positive and/or Gram-negative bacteria. In particular, the hydrogel is suitable for reducing biofilms of the Gram-positive pathogens Staphylococcus aureus (S. aureus) and of the Gram-negative pathogens Pseudomonas aeruginosa (P. aeruginosa). In a preferred embodiment, in a test according to ASTM E2871-13 or ASTM E2871-21, a reduction in colony-forming units (CFU) or a reduction in the number of pathogens by at least log₁₀=2, more preferably at least log₁₀=3, most preferably at least log₁₀=5, is achieved-especially with respect to S. aureus and/or P. aeruginosa. A surprisingly high efficacy is achieved in the reduction of biofilms containing solely Gram-positive pathogens. According to ASTM E2871-13 or ASTM E2871-21, a reduction in CFU by at least log₁₀=7, preferably by at least log₁₀=8 and most preferably by at least log₁₀=9 can be achieved here-especially with respect to S. aureus. The contact time between hydrogel or wound dressing of the invention and the biofilm here may be 24 h. A reduction in the biofilm by log₁₀=9 in a test according to ASTM E2871-13 can be equated with complete killing of the organisms present in the biofilm. Such a hydrogel preferably has an active ingredient content of 0.5% to 2.5% by weight. Further details of this can be taken from the working examples.

In the context of the present invention, the Ag₂Zn(EDTA) complex or complex compound is normally embedded into the hydrogel. By comparison with a coating of a hydrogel with an active ingredient, this leads to more stable integrity and more uniform release of the active ingredient. Furthermore, the micropores in the hydrogel remain clear and are not sealed, which leads to better mass transfer (release of moisture, absorption of exudate and pathogens). In a preferred embodiment, 95% by weight of the active ingredient used in the reaction mixture has been embedded into the hydrogel, more preferably 98% by weight and most preferably 99% by weight.

In one embodiment, the hydrogel of the invention contains 0.5% to 4% by weight of the Ag₂Zn(EDTA) complex or complex compound, preferably 0.5% to 2.5% by weight of Ag₂Zn(EDTA), more preferably 0.5% to 2% by weight of Ag₂Zn(EDTA) and most preferably 1% to 1.5% by weight of Ag₂Zn(EDTA). The concentration of Ag₂Zn(EDTA) in the hydrogel can be adjusted via the production process of the invention.

Measurements have shown that the active ingredient is released from the hydrogel to the wound in a greater amount than would have been expected. It has been found that, surprisingly, the active ingredient—in addition to its efficacy against bacteria and biofilms—additionally also enhances the release of moisture from the hydrogel layer of the invention. This means that a hydrogel that has been produced by the process of the invention but without active ingredient releases less moisture to a wound than an active ingredient-containing hydrogel of the invention. This effect is attributed to the fact that the active ingredient affects the crosslinking within the hydrogel matrix and hence also the tendency of the hydrogel to release water or moisture.

In one embodiment, the release of moisture from the hydrogels of the invention is at least 5 mg per square centimeter and day. The release of moisture increases with increasing active ingredient content. Preferably, the release of moisture is at least 8 mg per cm² and day, more preferably at least 15 mg per cm² and day and most preferably at least 18 mg per cm² and day. Hydrogels that correspond to the hydrogels of the invention in their structure but do not contain any active ingredient show a release of moisture of less than 5 mg per cm² and day. The release of moisture can be set here relative to a filter paper, which means that the measured moisture released is released to the filter paper. The filter paper may be a conventional laboratory filter paper having a diameter of 5 cm. The laboratory filter paper may have an average pore diameter of 15-20 μm and/or an average filtration rate of 35 and 37 seconds. The release of moisture is associated with release of active ingredient. The release of moisture can be determined by the method described in the present document (see example 1.4). In this context, the invention encompasses the use of an Ag₂Zn(EDTA) complex in a hydrogel in order to adjust or to enhance the release of moisture from the hydrogel. It holds true here that the release of moisture is at least 5 mg per cm² and day over and above an active ingredient content of 0.5% by weight, at least 15 mg per cm² and day over and above an active ingredient content of 1% by weight, and at least 18 mg per cm² and day over and above an active ingredient content of 1.5% by weight.

The active ingredient released in this way is capable of displaying a multiple effect. It counters harmful effects that are caused by bacteria (infection, biofilms, inflammation), and secondly improves wound healing by supplying the wound with moisture and cleaning it by a cleaning effect. By virtue of the cleaning effect, it is possible to remove or at least dilute substances that are harmful or present in excess and optionally absorb them into an absorbing layer of the wound dressing of the invention.

Hydrogels of the invention are preferably part of a wound dressing. In the context of the present invention, a wound dressing means a product suitable for application to a wound and for provision in ready-to-use form. Measurements have shown that hydrogels of the invention having a water content over and above 60% by weight show particularly effective release of the complex present as active ingredient in the hydrogel. Such hydrogels and wound dressings comprising them are preferred embodiments of the invention and are particularly suitable for reducing or fully dissolving biofilms in wounds.

In a preferred embodiment, wound dressings of the invention comprise at least the hydrogel of the invention as wound contact layer and a carrier layer which is on the opposite side from the wound contact layer and optionally comprises a circumferential adhesive region.

The wound contact layer offers various benefits. These include particularly tissue-conserving detachment of the wound dressing of the invention being ensured when the dressing is changed. In addition, the wound contact layer can disinfect the wound, prevent or reduce bacterial colonization of the wound, supply the wound with moisture, accelerate the healing process, have wound margin care properties, prevent skin irritation and have a nonstick effect. The hydrogels of the invention are of excellent suitability as wound contact layer. They do not stick to the wound and prevent granulation tissue from growing into the wound dressing.

The carrier layer especially has the function of preventing drying out of the hydrogel on the side remote from the wound. However, a low level of release of moisture or water vapor through the carrier layer may remain possible.

Wound dressings of the invention may comprise the following layers: a carrier layer, a hydrogel layer according to the present invention, and preferably an absorbing layer disposed between the hydrogel layer and the carrier layer.

Accordingly, the wound dressing advantageously further comprises, between the wound contact layer and the carrier layer, an absorbing layer, preferably in the form of an absorbing foam, more preferably in the form of an absorbing polyurethane foam. For example, a wound-facing side of the polyurethane foam may have been coated with the hydrogel, while a side of the polyurethane foam remote from the wound is bonded to the carrier layer. In addition, in this embodiment, the hydrogel may be configured as a meshlike wound contact layer. In this way, it is possible to improve the passage of wound exudate into the additional absorbing layer.

The active ingredient prevents bacteria that have migrated or been absorbed into the wound dressing from being able to multiply therein. Since bacteria from the wound must pass through the hydrogel layer (wound contact layer) before they reach an absorbing layer, they inevitably come into contact with the active ingredient. In this way, subsequent multiplication of the microbes in the absorbing layer is prevented.

Carrier layers used may especially be polymer films or polymer foams, preferably films or foams that are manufactured from polyurethane, polyetherurethane, polyesterurethane, polyether-polyamide copolymers, polyacrylate or polymethacrylate. In particular, a suitable carrier layer is a water-impermeable and water vapor-permeable polyurethane film or a water-impermeable and water vapor-permeable polyurethane foam. In particularly preferred embodiments, these films have a moisture-tight circumferential adhesive region. This region ensures that the wound system can be applied and fixed to its intended site. Furthermore, it is ensured that no liquid can escape between the film and the skin adjoining the wound. Possible adhesives are acrylic adhesives or acrylic-based adhesives. These ensure particularly strong adhesion. Possible further adhesives are silicone adhesives or silicone-based adhesives. These enable atraumatic adhesion and are particularly suitable in the case of damaged or sensitive skin and frequent changing of dressings. The circumferential adhesive region may comprise the adhesives listed in this section as adhesive layer.

Possible arrangements of the different layers in multilayer wound dressings of the invention are described, for example, in WO 2010/000450, which is hereby fully incorporated by reference.

In addition, the wound dressing may also comprise further layers as well as the absorbing layer and the carrier layer, for example one or more barrier layers and/or one or more distributor layers.

It is customary to provide the wound dressing with a covering film/peelable film on the wound contact side. It is thus possible to protect the wound dressing from contamination during storage and to avoid a loss of moisture. The covering film should be removed prior to application.

The wound dressing may have a rectangular or essentially square basic shape. Preference is given here to a size range of 8 cm×8 cm up to 20 cm×20 cm. The thickness of the wound dressing is preferably less than 2 cm, with a possible foam layer preferably having a thickness between 0.1 cm and 1.8 cm, preferably between 0.3 cm and 0.8 cm.

Wound dressings have additionally been found to be particularly advantageous executions, comprising a hydrogel matrix or a wound contact layer composed of hydrogel, the layer thickness of which is 0.1 to 5.0 mm. In particular, a wound dressing of the invention thus includes a wound contact layer having a layer thickness of 0.1 to 5.0 mm, in particular of 0.5 to 5.0 mm and most preferably of 0.5 to 3.0 mm. Wound dressings having such layer thicknesses show the ability to absorb a wound exudate released by a wound and to pass it on to the absorbing layer. These layer thicknesses may be the same right across the wound contact layer or assume different values in different regions of the wound contact layer.

The hydrogels of the invention are suitable for the treatment of wounds. The present invention therefore also encompasses hydrogels of the invention for treatment of wounds. In particular, the present invention encompasses hydrogels for treatment of infected wounds, chronic wounds such as decubitus ulcers, pressure ulcers, pressure sores, ulcus cruris venosum, venous ulcers, ulcus cruris arteriosum, arterial ulcers, wounds resulting from diabetic foot syndrome, neuropathic ulcers, but also wounds resulting from autoimmune diseases or from tumors (ulcerating tumors) or from radiation damage in the case of tumor therapy.

In this context, the invention also encompasses a method of treating wounds—especially infected wounds—and of biofilms in wounds, comprising the following steps:
1) applying the hydrogel of the invention or the wound dressing of the invention to a wound or to an infected wound or biofilm-colonized wound,
2) optionally fixing or covering the hydrogel or the wound dressing of the invention, where the fixing or covering can be effected with the aid of a circumferential adhesive region (in the case of a wound dressing), or with the aid of adhesive strips, a bandage and/or a compress, and
3) optionally changing the hydrogel or the wound dressing after 2 to 7 days, preferably after 3 to 6 days.

Hydrogels of the invention, or wound dressings comprising them, are suitable for phase-appropriate wound therapy, especially for therapy of wounds in the granulation phase and/or the epithelialization phase.

### EXAMPLES

The present invention is described in detail by the nonlimiting examples that follow.

1. Hydrogels without Foam

### Example 1.1 Production of the Hydrogels without Active Ingredient

Hydrogel prototypes were produced in two different ways, mainly manually or with a gel casting system. The main sample preparation was conducted with the B100 gel system which is sold by bdtronic and enables dynamic control and combination of two components with establishment of a precise mixing ratio. It is possible to produce identical prototypes having the same thickness and same weight/area ratio.

For product optimization, hydrogels were additionally produced manually under laboratory conditions. Because of the different reaction rate at different mixing ratios, some samples had to be combined rapidly and distributed directly into petri dishes in order to avoid curing while stirring. By contrast with the gel machine, this method was more suitable for small amounts of hydrogels with different concentrations.

The following table shows the percentage distribution of the constituents for hydrogel formation:

TABLE 1

| | | Proportion in the | Solution content |
|---|---|---|---|
| Solution | Component | hydrogel [% by wt.] | [% by wt.] |
| A | glycerol | 16.88 | 87 |
| | Jeffamine | 7.58 | |
| | water | 62.53 | |
| B | Aquapol | 13 | 13 |

The B100 gel system consists of a dynamic two-component mixing head with material supply via two pneumatically controlled needle valves, the two reservoir vessels for the solution of alcohol-amine mixture and Aquapol, and the control unit with digital display. The system also has a compressed air processing unit. Solutions A and B (see table) were introduced into the two cartridges. In this filling operation, there is the risk of air being incorporated in the solutions. In order that this does not disrupt the casting process, the two solutions were left to stand for twelve hours for outgassing. The components were conveyed by external pumps via separate hoses to needle valves mounted on the mixing head. Since the two components react with one another here, the mixing time has to be kept below the gel formation time, since there would otherwise be hardening within the mixing head. In order to assure a constant mass flow rate, irrespective of the viscosity of the medium, the cartridges were charged with compressed air. The pressure established was 1.5 bar on both sides. In order to avoid moisture in the system, the compressed air was dried beforehand in a silica gel filter. The gel produced was expelled via the outlet nozzle in the mixing head and introduced into plastic petri dishes. The maximum metering output was 3.50 g/s.

Since the system is a two-component mixing system, prior to gel production, a batch solution of Jeffamine, glycerol and water (solution A) was produced. Since the weight ratio between Jeffamine and Aquapol is responsible for gel formation, exact adjustment of the correct mixing ratio is indispensable. It was found by empirical analyses that, in the case of active ingredient-free gels, a mass ratio of mass of Aquapol to mass of Jeffamine=1.7 is optimal. Mass ratios of 1.3 to 1.8 likewise gave satisfactory results.

In addition, it was found that, in the case of addition of the active ingredient to the hydrogels, said weight ratio had to be adjusted in order to obtain optimal results. This is set out in detail in the section below.

### Example 1.2 Production of Gels Having Different Active Ingredient Contents

Hydrogels were produced by the method described in example 1, but with a different active ingredient content. For this purpose, a 2.5% by weight stock solution of the active ingredient in water was made up, which was added to the gels in different proportions. It was found that, unexpectedly, the active ingredient added reduced the degree of crosslinking of the resulting hydrogels. Further analyses showed that, with rising active ingredient concentration, the results were improved by an increasing increase in the proportion of isocyanate-terminated prepolymer relative to the amine-terminated prepolymer. Controlled addition of the isocyanate-terminated prepolymer afforded hydrogels having excellent stability. The composition of the different hydrogels is given in table 2 below, again with inclusion of the active ingredient-free gel from table 1 for comparison. The proportion in the reaction mixture is reported in each case in percent by weight.

TABLE 2

| | | Proportion | Proportion | Proportion | Proportion |
|---|---|---|---|---|---|
| | | in the | in the | in the | in the |
| | | reaction | reaction | reaction | reaction |
| | | mixture for | mixture for | mixture for | mixture for |
| | | a gel | a gel with | a gel with | a gel with |
| | | without | 0.5% by wt. | 1% by wt. of | 1.5% by wt. |
| | | active | of active | active | of active |
| Solution | Component | ingredient | ingredient | ingredient | ingredient |
| A | glycerol | 16.88 | 14.57 | 13.36 | 11.16 |
| | Jeffamine | 7.58 | 7.58 | 7.58 | 7.58 |
| | H₂O | 62.53 | 42.54 | 22.49 | 0 |
| | Aqueous | 0 | 19.99 | 40.08 | 62.53 |
| | active | | | | |
| | ingredient | | | | |
| | stock soln. | | | | |
| | 2.5% | | | | |
| B | Aquapol | 13 | 15.31 | 16.52 | 18.72 |
| Aquapol/ | | 1.7 | 2.02 | 2.18 | 2.47 |
| Jeffamine | | | | | |
| ratio | | | | | |

For production of solution A, the appropriate amount of Jeffamine was heated to 50° C. in a water bath for 30 min. Subsequently, the specified amount of water was added. The components were mixed with the aid of a magnetic stirrer, in the course of which the necessary amount of active ingredient stock solution was added. Solutions A and B were introduced into the B100 system, and the gels were produced by mixing the two solutions with the aid of the system.

The hydrogels from tables 1 and 2 were used for the experiments described hereinafter.

### Example 1.3 Kinetics of the Production of the Hydrogels

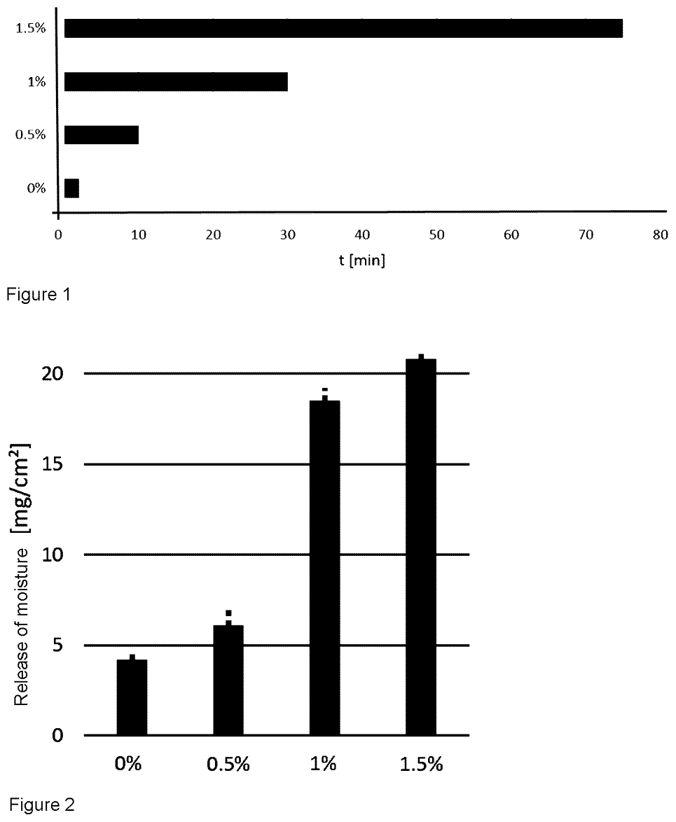

It has been found that the reactions for production of the hydrogels have different reaction rates. The respective reaction rates depended on the active ingredient content. The reaction was considered to be complete as soon as what is called the gel point had been attained and a nontacky gel surface had formed. The results are shown in FIG. 1.

While a reference gel without active ingredient (composition according to tab. 1) took about 2 min to reach the gel point, the time was about 10 min with an active ingredient content of 0.5%, about 30 min with 1%, and about 75 min with 1.5%.

### Example 1.4 Moisture Release by the Hydrogels

The release of moisture to filter paper for hydrogels of the invention with different active ingredient contents was measured. A gel without active ingredient (composition according to tab. 1) was incorporated into the measurement as reference.

For the measurement, samples having a diameter of 2.5 cm were punched out, and their starting weight was determined. Subsequently, the samples were placed into petri dishes lined with filter paper. Petri dishes and filter paper had a diameter of 5 cm and were likewise weighed beforehand. The petri dishes were sealed and incubated at a temperature of 37° C. After 24 h, the samples and filter paper+petri dish were weighed independently. The release of moisture was determined by the following formula:

$$Release of moisture = \frac{( mt - m 0 ) \times 1000}{A}$$

$$mt = weight of petri dish and filter paper after 24 h [ g ]$$

$$m 0 = starting weight of petri dish and filter paper [ g ]$$

$$A = area of the filter paper [ {cm}^{2} ]$$

The results are shown in the following table and additionally as a bar diagram in FIG. 2:

TABLE 3

| Active ingredient concentration [%] | Release of moisture [mg/cm²] |
|---|---|
| 0 | <5 |
| 0.5 | 6.1 |
| 1 | 18.5 |
| 1.5 | 20.8 |

### Example 1.5 Active Ingredient Release from the Hydrogels

### Release of Silver and Zinc

Gel samples having an active ingredient concentration of 1.5% were tested to determine the release. Optical emission spectroscopy with inductively coupled plasma (ICP-OES) was used for the analysis of trace elements in the concentration range of mg/l to μg/l. This method permits simultaneous determination of all metals and some nonmetals from acidified aqueous solutions up to a content of about 10 g/l.

For determination of the ion release of silver and zinc, hydrogel samples having a weight of one gram were transferred into a sample vessel, and 10 ml of demineralized water was added. Fifteen samples were made up and incubated on a shaker at 180 rpm at room temperature. After 4, 24, 48 and 72 hours and 7 days, 3 ml of the solution for each sample was pipetted into vials. These samples were analyzed according to EN ISO 17294-2 (E29). This was done by subjecting the samples to pressure digestion with nitric acid in Teflon vessels. The resulting clear solution was diluted to a final concentration of 10-50 g/l and analyzed. In ICP-OES analysis, the sample solution was introduced via a pneumatic atomizer system into an inductively coupled argon plasma. At a temperature of 5000-7000 K in the plasma, the elements were atomized and induced to emit light. The emitted light was detected in the simultaneous analysis by a polychromator divided into element-specific wavelengths. In order to quantitatively determine the element content of a solution, the instrument was calibrated with synthetic solutions having known content. Analysis of the migration of zinc ions in 1.5% hydrogels into H₂O showed an almost linear rise from 0.07 g/l after 24 hours to 0.13 g/l after 7 days. It can therefore be assumed that the ions are released constantly.

By contrast, analysis of the released silver ions in the same medium showed a significant drop in release after about one day, until a constantly low level was attained after about 72 hours. While the values were constant at 0.19 g/l over a period of 24 hours, the detectable amount of silver ions fell to 0.02 g/l after 7 days.

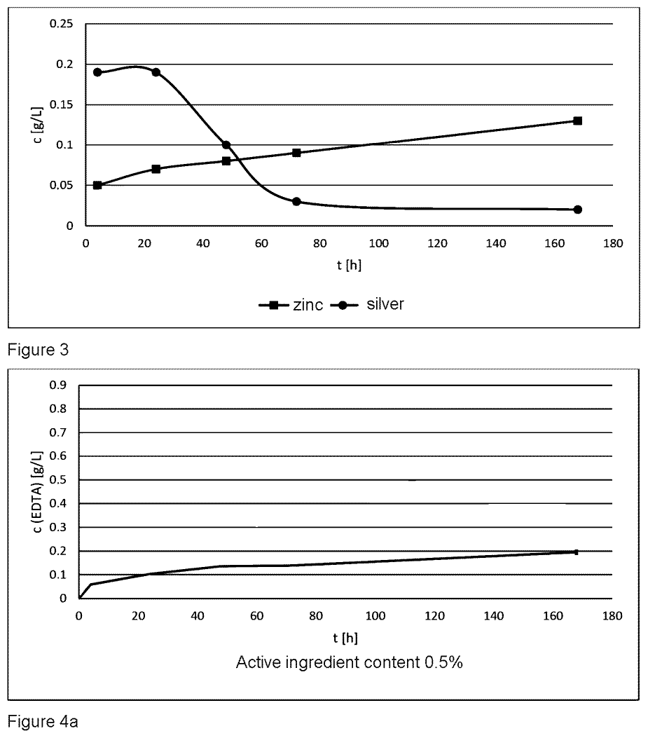

The results are shown in FIG. 3.

### Release of EDTA

The release of EDTA was determined by UV/VIS spectroscopy. In order to examine the migration of the EDTA present in the hydrogel into an aqueous solution, the eluate was analyzed with a photoLab® S6 photometer. For this purpose, the absorption maximum of sodium-EDTA in an aqueous solution was estimated. For the development of a calibration curve, a serial dilution of NaEDTA concentrations in the range of the calculated maximum exposure limits of hydrogels was established. This covered the concentration range from 0 to 1 g/l.

In order first to ascertain the absorption maximum of the EDTA complex present in the hydrogels, a UV/VIS measurement on a 1 molar sodium-EDTA complex solution (NaEDTA) was first conducted. This gave a maximum at a wavelength of \tilde{v}=605 nm.

For the determination of the release, hydrogel samples having a weight of 1 g were transferred into sample vessels, and 10 ml of demineralized water was added to each. The samples were incubated on a shaker at 180 rpm at room temperature. After 4, 24, 48 and 72 hours and 7 days, 3 ml of the eluates were pipetted into polystyrene cuvettes, and absorption in % was measured at a wavelength of 605 nm.

The measurements of absorption on the hydrogels of the invention at a wavelength of 605 nm showed a trend toward increasing absorption proportionally to the rising active ingredient concentration. While the hydrogels had absorption of 0.034% with a concentration of 0.5% after 24 hours, this value almost doubled (0.064%) within 7 days. Doubling of the absorption was also observed in the case of hydrogels with 1% active ingredient, specifically from 0.077% after 24 hours to 0.134% after 7 days. 1.5% of hydrogels showed an absorption of 0.072% after 24 hours and 0.249% after 7 days.

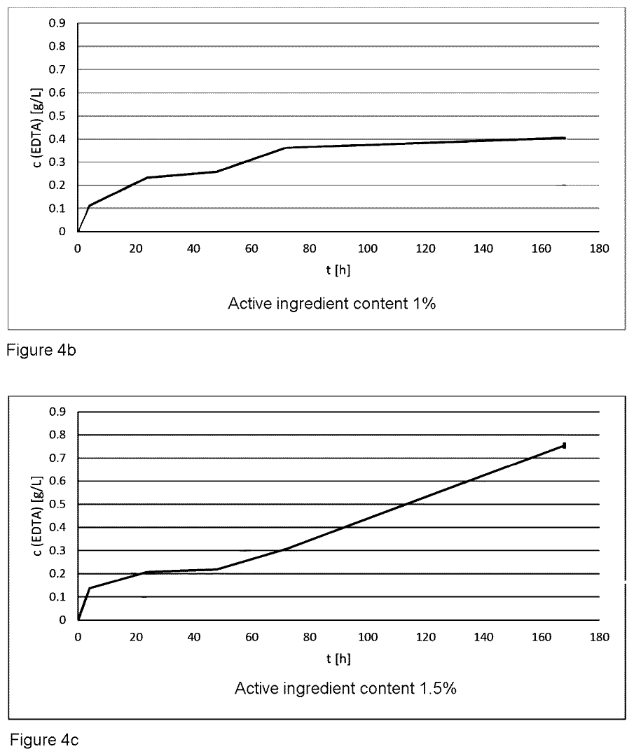

Using the calibration curve established above (absorption as a function of EDTA concentration), the maximum EDTA concentration transferred to the medium after 7 days was 0.194 g/l for 0.5% hydrogels, 0.406 g/l for 1% hydrogels, and 0.754 g/l for 1.5% hydrogels. The measurements are shown as curve diagrams in FIG. 4a (active ingredient content 0.5%), 4b (active ingredient content 1%) and 4c (active ingredient content 1.5%).

As apparent from the figures, the concentration of the EDTA complexes released was proportional to the active ingredient content in the hydrogels.

### Example 1.6 Antimicrobial Action of the Hydrogels Against Biofilms

In order to determine the antimicrobial action of hydrogels of the invention, the efficacy against biofilms of the two human-pathogenic bacteria S. aureus (strain according to ATCC deposit 6538) and P. aeruginosa (strain according to ATCC deposit 15442) was analyzed. In this context, S. aureus represented the Gram-positive bacteria, and P. aeruginosa the Gram-negative bacteria. The test was effected according to the publicly accessible standard protocol ASTM E2871-13. CDC bioreactors (Center for Disease Control) were used to create the biofilms. Multiple rods are embedded in the lid of these bioreactors, which dip into the nutrient solution in use. The rods have cutouts capable of accommodating special platelets-so-called coupons. During incubation in the bioreactor, biofilms are formed on the coupons. The anti-biofilm action of the hydrogels of the invention was tested on these biofilms.

First of all, an individual colony of each species was inoculated in 10 ml of TBS (Tris-buffered saline) and incubated on an agitator plate at 37° C. and 125 rpm overnight.

The overnight culture of P. aeruginosa was adjusted to 10⁸ CFU (colony-forming units) per ml. Subsequently, this was used to seed a CDC bioreactor by adding 1 ml to 300 ml of TSB. The bioreactor was incubated in the batch phase at 37° C. and 80 rpm on an agitator plate for 24 h.

The overnight culture of S. aureus was centrifuged for three minutes, the supernatant was discarded, and the cell pellet was resuspended in 1 ml of TSB. The resuspended pellet was used to inoculate the CDC bioreactor by adding it to 300 ml of TSB. The bioreactor was incubated in the batch phase at 37° C. and 80 rpm on an agitator plate for 24 h.

After the 24 h had elapsed, biofilms had formed. The rods were pulled out of the bioreactor and washed twice with PBS. The coupons were wrapped in hydrogels that had previously been cut to a size of 2.5×5 cm. Three coupons per batch were used. The wrapped coupons were placed into 12-well plates and incubated at room temperature (P. aeruginosa) or 37° C. (S. aureus) for 24 h.

The next day, the coupons were removed from the wells, added to 10 ml of Dey Engley neutralizing broth and treated with ultrasound for 30 min.

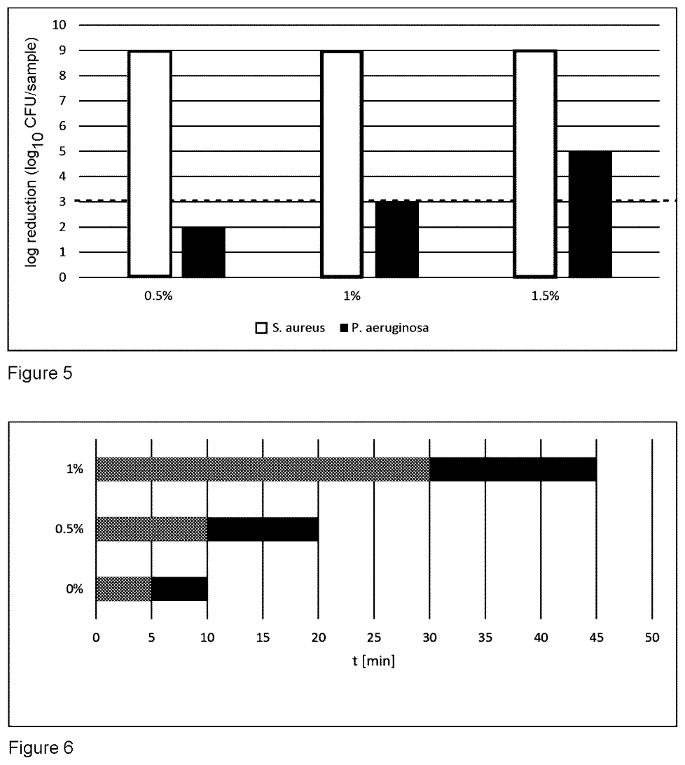

Subsequently, the samples were shaken on a vortex device and distributed on 96-well plates. Each sample was serially diluted with PBS in a ratio of 1:10, and 20 μl of each dilution was distributed on TSA plates (tryptone soy agar), in duplicate in each case. The samples were incubated at 37° C. overnight, and the colonies were counted the next day. In this way, the total number of viable bacteria was determined. The results for hydrogels having an active ingredient content of 0.5%, 1% and 1.5% are shown as a diagram in FIG. 5.

With respect to biofilms of S. aureus, hydrogels having all three tested active ingredient concentrations showed a reduction in the colony-forming units (CFU) of log₁₀=9 and an hence extremely high efficacy. Since a higher active ingredient concentration did not result in any further reduction in the colony-forming units, it can be assumed that even the lowest active ingredient concentration of 0.5% resulted in complete killing of all bacteria present in the biofilm. With respect to biofilms of P. aeruginosa, the antimicrobial effect increased with rising active ingredient concentration. Hydrogels having an active ingredient content of 0.5% showed a logarithmic reduction of log₁₀=2, a reduction of log₁₀=3 at an active ingredient content of 1%, and of log₁₀=5 at 1.5%. It can thus be assumed that the effect of the hydrogels of the invention with respect to P. aeruginosa (and possibly with respect to Gram-negative bacteria in general) is dose-dependent and increases further with rising active ingredient concentration.

### 2. Hydrogels on Polyurethane Foam

### Example 2.1 Production of Active Ingredient-Containing Hydrogels on PU Foam

Hydrogels having an active ingredient content of 0.5% and 1% by weight were bonded to a polyurethane foam during their production. An additional reference used here was a hydrogel without active ingredient. In the tests conducted, it was found that the juncture of application of the hydrogel to the PU foam is crucial. When the hydrogels are contacted with the PU foam, the hydrogel matrix must still have sufficient crosslinking potential. On the other hand, the reaction mixture must not be applied to the foam too early, since it is otherwise absorbed by the foam. Even though the optimal juncture for the reference gel was after the gelation time, it has been found that the gelation time is the optimal juncture for the active ingredient-containing gels. The results are shown in FIG. 6: The hatched region of the diagram bars shows the period of time from the start of the reaction up to the time at which the gel was contacted with the PU foam. The black region of the diagram bars shows the period of time in which the gels bonded to the PU foam. For this purpose, foams of corresponding size to the gel were introduced into the casting mold. The bonding operation was considered to be complete as soon as it was possible to remove the gel-foam composite from the casting mold without separation or damage. 10 min was required in the case of an active ingredient content of 0.5% by weight, and 15 min was required in the case of an active ingredient content of 1% by weight, before gel and foam had bonded to one another. PU foams that were stably coated with hydrogels of the invention and could be removed from the casting mold were the result.

### Example 2.2 Absorption Capacity of Hydrogels on PU Foam

The absorption capacity of the coated PU foam was determined by punching samples of size 2.5×2.5 cm out of the hydrogel-coated foams. Each sample was weighed and placed into a glass beaker containing demineralized water. After 24 hours in each case, the samples were removed, dried with the aid of wood pulp paper and weighed. In order to determine the absorption capacity for each sample, the following formula was used:

$$absorption capacity = ( final weight after 24 h - starting weight ) / starting weight$$

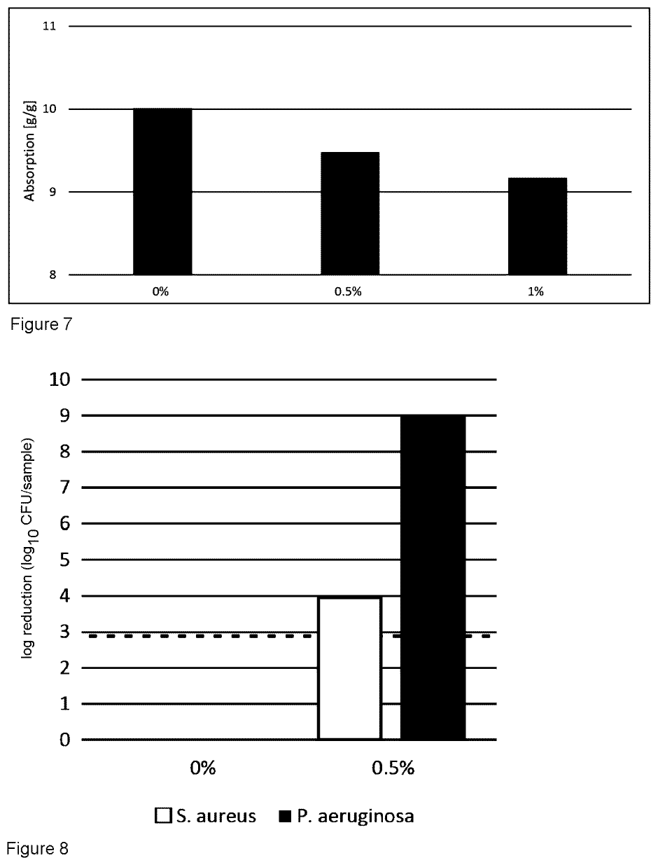

After 24 hours, hydrogels having an active ingredient content of 0.5% on PU foam had an absorption of 9.47 g/g, whereas an absorption of 9.17 g/g was measured in the case of an active ingredient content of 1%. In the case of a reference gel on PU foam without active ingredient, the measurement was 10 g/g. A higher active ingredient content therefore appears to be associated with lower absorption. However, other measurements showed that a higher active ingredient content also simultaneously promotes the release of moisture from the gel, and so it can be suspected that the higher moisture release is associated with lower propensity to absorption. The results are shown as a diagram in FIG. 7.

### Example 2.3 Bond Strength of Hydrogels on PU Foam

The bond strength of the hydrogels bonded to PU foam was tested on steel. A tensile test machine that conformed to standard DIN EN ISO 7500-01 was used. All measurements took place under standardized conditions of 23° C. and 50% relative humidity. Samples of size 5×5 cm were punched out. The samples were attached by the reverse side (foam side) to a horizontally movable support by means of double-sided adhesive tape. The measurement was performed using a metal weight having a weight force of 0.245 N with a glass underside. The underside was cleaned with an ethanol-soaked pad before the start of the measurement. The weight was placed by the underside first on the hydrogel, and the measurement was conducted according to the following parameters:

TABLE 4

| | Pull-off speed | Contact time |
|---|---|---|
| Initial speed [mm/min] | [mm/min] | [s] |
| 100 | 400 | 2 |

After the contact time had elapsed, the tensile test machine was used to measure the force required to pull off the weight at a 90° angle. Five measurements were conducted for each active ingredient content, and the following average values were ascertained:

TABLE 5

| Active ingredient | |
|---|---|
| content [%] | Bond strength [N] |
| 0 | 0.767 |
| 0.5 | 0.834 |
| 1 | 0.867 |

As is apparent, bond strength surprisingly rose with increasing active ingredient concentration.

### Example 2.4 Antimicrobial Action of the Hydrogels on PU Foam Against Biofilms

The test for antibacterial efficacy of the hydrogels toward biofilms that was described further up was repeated. This time, hydrogels having an active ingredient concentration of 0% and 0.5% attached in the form of a coating on PU foam were tested. The results are shown in FIG. 8.

As apparent from the results, hydrogels on PU foam without active ingredient do not show any effect against biofilms of S. aureus or P. aeruginosa. By contrast, in the case of an active ingredient concentration of 0.5%, antibacterial action against the biofilms is measurable. For instance, the biofilm of S. aureus was reduced by a logarithm of log₁₀=4. In the case of the biofilm of P. aeruginosa, there was even a reduction of log₁₀=9. As discussed in the results for the antibacterial action of hydrogels without foam that were presented above, in the case of a reduction of log₁₀=9, it can be assumed that all bacteria within the biofilm have been killed.

Proceeding from the results presented here, it may be suspected that hydrogels of the invention as a coating of PU foam display particularly high efficacy against biofilms of Gram-negative bacteria such as P. aeruginosa, and hydrogels of the invention without foam achieve particularly high efficacy against biofilms of Gram-positive bacteria such as S. aureus.

### The Invention Preferably Further Encompasses:

A hydrogel of the invention, wherein the release of moisture from the hydrogel in a period of 24 h is at least 5 mg per square centimeter, preferably 10 mg per square centimeter, more preferably 15 mg per square centimeter and most preferably 20 mg per square centimeter. The use of an Ag₂Zn(EDTA) complex in a hydrogel in order to adjust or to enhance the bond strength of the hydrogel.

The use of an Ag₂Zn(EDTA) complex in a hydrogel in order to adjust or to enhance the release of moisture from the hydrogel.

1. A process for producing a biofilm-reducing hydrogel for medical purposes, comprising the following steps:
i. providing an isocyanate-terminated prepolymer containing polyalkylene oxide units,
ii. providing an amine-terminated prepolymer containing polyalkylene oxide units,
iii. dissolving the substance provided in step ii in a water-containing liquid in order to obtain an aqueous formulation,
iv. mixing the aqueous formulation and a solution, containing an Ag₂Zn(EDTA) complex, in order to obtain a starting solution,
v. combining the starting solution and the isocyanate-terminated prepolymer to give a reaction mixture, whereby the reaction mixture is converted by polymerization to the hydrogel,
wherein the pH of the solution from step iv is at least 9, and wherein the reaction mixture from step v does not contain any acrylic acid or polyacrylic acid.

2. The process as claimed in claim 1, wherein the mass ratio of the isocyanate-terminated prepolymer to the amine-terminated prepolymer is between 1.3 and 3.2.

3. The process as claimed in claim 1, wherein the water-containing liquid in step iii contains glycerol.

4. The process as claimed in claim 1, wherein the reaction mixture contains 10% to 30% by weight of glycerol.

5. The process as claimed in claim 1, wherein the pH of the solution from step iv is adjusted by means of ammonia, sodium hydroxide or acetic acid.

6. The process as claimed in claim 1, wherein the reaction mixture does not contain any triethanolamine, any chloride ions, and/or any chlorine salts.

7. The process as claimed in claim 1, wherein the reaction mixture from step v contains 0.5% to 4% by weight of the Ag₂Zn(EDTA) complex.

8. The process as claimed in claim 1, wherein the sum total of the masses of amine-terminated prepolymer and isocyanate-terminated prepolymer is 10% to 30% by weight of the reaction mixture.

9. A hydrogel having biofilm-reducing properties, obtainable by the process as claimed in claim 1.

10. The hydrogel as claimed in claim 9, wherein the hydrogel has a water content of 40% to 65% by weight.

11. The hydrogel as claimed in claim 9, wherein the hydrogel has a pH of 6.5 to 9.5.

12. The hydrogel as claimed in claim 9, wherein the hydrogel reduces the number of bacterial cells of S. aureus or P. aeruginosa within a biofilm during a test according to ASTM E2871-13 and over a contact time of 24 h by at least log₁₀=2.

13. The hydrogel as claimed in claim 9, wherein the hydrogel contains 0.5% to 4% by weight of the Ag₂Zn(EDTA) complex.

14. The hydrogel as claimed in claim 9, wherein the release of moisture from the hydrogel is at least 5 mg per square centimeter and day.

15. A wound dressing comprising: a hydrogel as claimed in claim 9 as a wound contact layer, and a carrier layer opposite the wound contact layer, wherein the carrier layer optionally comprises a circumferential adhesive region.

16. The wound dressing as claimed in claim 15, further comprising an absorbing foam between the wound contact layer and the carrier layer.

17. The process as claimed in claim 1, the solution from step iv is an aqueous solution.

18. The process as claimed in claim 1, wherein the pH of the solution from step iv is from 10 to 12.

19. The hydrogel as claimed in claim 9, wherein the hydrogel has a pH of 6.5 to 8.

20. The wound dressing as claimed in claim 15, wherein the carrier layer is an absorbing polyurethane foam.
